(19) United States
(12) Patent Application Publication
(10) Pub. No.: US 2026/0033909 A1
(43) Pub. Date: Feb. 5, 2026

(54) SURGICAL MEDIA SYSTEM

(71) Applicant: Johnson & Johnson Surgical Vision, Inc., Irvine, CA (US)

(72) Inventors: Linyi Xia, Irvine, CA (US); Edith Fung, Diamond Bar, CA (US)

(21) Appl. No.: 18/791,214

(22) Filed: Jul. 31, 2024

(51) Int. Cl. A61B 90/00; A61B 17/00; A61F 9/007; G06T 5/60; G06T 11/00; H04N 7/18

(52) U.S. Cl. CPC A61B 90/361; A61F 9/00745; G06T 5/60; G06T 11/00; A61B 2017/00199; A61B 2017/00973; A61B 2090/364; A61B 2090/373; G06T 2207/10016; G06T 2207/20092; G06T 2207/30041

(57) ABSTRACT

Systems and methods for streaming video content relating to an ophthalmic surgical procedure are provided herein. A method includes receiving video content captured from a video source during the ophthalmic surgical procedure and receiving overlay information. A method includes sending at least a portion of the video content and the overlay information to a processing device configured to provide a video stream enhancement application, and receiving image enhancement information from the processing device. A method includes processing the video content based on the received image enhancement information to produce an enhanced video stream including an overlay representing at least at portion of the overlay information and sending, using a communication interface, the enhanced video stream to one or more receive devices configured to display the enhanced video stream.

### FIELD OF INVENTION

This invention generally relates to the field of ocular surgery and, more specifically, to systems and methods for receiving, processing, transmitting, and displaying a surgical video feed in conjunction with other data.

### BACKGROUND

Systems for use in ocular surgery may provide functionality for processing and displaying surgical data, including surgical parameters, video data, sensor data, and metadata associated with the procedure. Processing and display of surgical data in real time may be necessary, as surgeons continuously make decisions while conducting the surgical procedure. Because the surgical data may provide cues to the surgeon and operating room personnel as to how to conduct the procedure and how to adjust and refine settings and surgical parameters, the timely processing and presentation of surgical data is critical to a safe and effective procedure.

The phacoemulsification system, which is used in cataract surgery, is one example of a system that may benefit from such functionality. In a phacoemulsification procedure, the surgeon uses vacuum/aspiration and irrigation flow with ultrasound power to break up and dispose of cataract tissue. An external display provides a video stream of the surgeon's view from a surgical microscope along with a data stream of surgical settings and status data from the system superimposed on the video stream in an overlay. The combined stream with overlay information is used by operating room staff to monitor the system and procedures and to assist the surgeon during cataract surgery.

In addition to supporting operating room personnel during a live procedure, some systems provide functionality for storing and reviewing previously captured surgical data. For example, digital video capture and data storage may allow surgeons to record images during the course of the surgical procedure as captured by a camera used in conjunction with the surgical microscope and may allow surgeons to view or display captured video and/or surgical data subsequent to the surgical procedure. Systems may also be capable of capturing surgical parameters and sensor data during the procedure along with timestamps that allow for analysis in conjunction with the captured video feed.

### BRIEF DESCRIPTION OF THE DRAWINGS

FIG. 1 is a schematic, pictorial illustration of an example of a phacoemulsification system;

FIG. 2 is a block diagram illustrating an example of a surgical media system;

FIG. 3 is a diagram illustrating the architecture of an exemplary system in which the surgical media system operates; and

FIG. 4 is a flow diagram illustrating an exemplary method for processing of video content and overlay information.

### DETAILED DESCRIPTION OF THE PREFERRED EXAMPLE(S)

The surgical environment typically includes a variety of equipment such as a surgical console, accessories, implements, video sources (including, for example, microscopes and/or cameras), displays, storage devices, and interface hardware. The environment may include a surgical media system that interfaces with other equipment within the system and processes video streams, surgical data, and overlay information to be displayed. As is described in further detail below, the surgical media system, surgical console, accessories, implements, video sources, displays, storage devices, and/or interface hardware may also be connected to or communicate with devices outside of the surgical environment. For example, devices located outside of the surgical environment may be configured to receive, process, analyze, or display video streams, surgical data, and/or overlay information to be displayed.

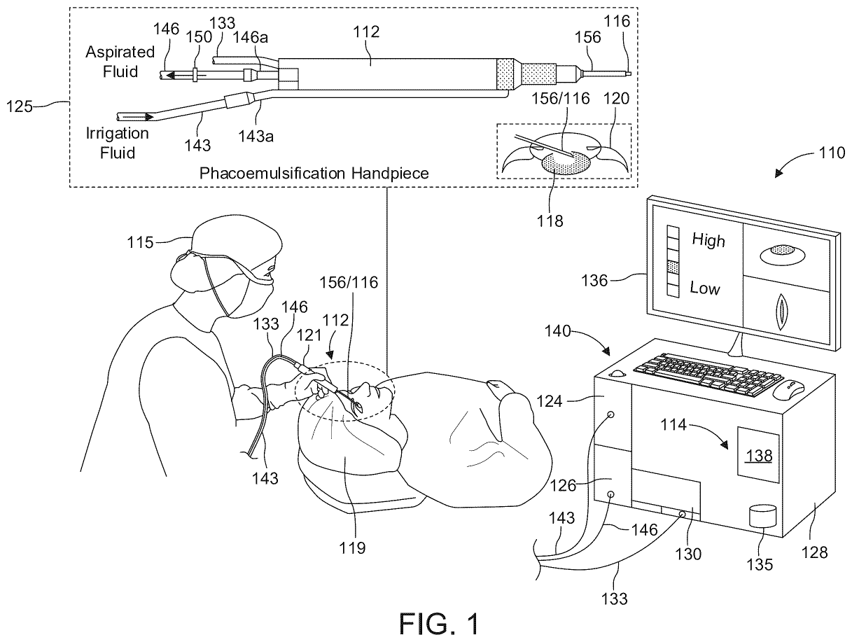

FIG. 1 is a schematic, pictorial view, along with an orthographic side view, of a phacoemulsification system 110, in accordance with an example of the present disclosure. As seen in the pictorial view of phacoemulsification system 110, and in the schematic side view inset 125, a phacoemulsification probe 112 (e.g., a handpiece) comprises a distal end 111 comprising a needle 116 and a coaxial irrigation sleeve 156 that at least partially surrounds needle 116 and creates a fluid pathway between the external wall of the needle and the internal wall of the irrigation sleeve, where needle 116 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 116 is configured for insertion into a lens capsule 118 of an eye 120 of a patient 119 by a physician 115 to remove a cataract. While needle 116 (and irrigation sleeve 156) are shown in inset 125 as a straight object, any suitable needle may be used with phacoemulsification probe 112, for example, a curved or bent tip needle commercially available from Johnson & Johnson Surgical Vision, Inc., Irvine, CA, USA.

In the shown example, during the phacoemulsification procedure, an irrigation pump 124, comprised in a console 128, pumps irrigation fluid from an irrigation reservoir (not shown) to irrigation sleeve 156 to irrigate the eye. The fluid is pumped via an irrigation tubing line 143 running from console 128 to an irrigation channel 143a of probe 112. In another example, pump 124 may be coupled with, or replaced by, a gravity-fed irrigation source such as a balanced salt solution bottle/bag. Fluid and waste matter (e.g., emulsified parts of the cataract) are aspirated via hollow needle 116 to a collection receptacle (not shown) by a processor-controlled aspiration pump 126, also comprised in console 128, using aspiration tubing line 146 running from aspiration channel 146a of probe 112 to console 128.

In the shown example, probe 112 includes an irrigation sensor 127 (e.g., pressure sensor) coupled with irrigation channel 143a and an aspiration sensor 123 (e.g., vacuum sensor) coupled with an aspiration channel 146a. Irrigation sensor 127 may be positioned anywhere along tubing line 143 or channel 143a. Likewise, aspiration sensor 123 may be positioned anywhere along tubing line 146 or channel 146a.

Channels 143a and 146a are coupled respectively with irrigation line 143 and aspiration line 146. Pumps 124 and 126 may be any pump known in the art (e.g., peristaltic pump, progressive cavity pump). Using sensors (e.g., as indicated by sensors 127 and/or 123), a processor 138 controls a flow rate of irrigation pump 124 and/or aspiration pump 126 to maintain IOP within prespecified limits.

The phacoemulsification system 110 includes an anti-vacuum surge (AVS) module 150, coupled with the probe 112, which prevents a sudden vacuum increase being transferred to the eye in case of an occlusion break. In some cases, typically to protect against a vacuum surge hazard, the system activates AVS module 150 (seen in inset 125) to disconnect aspiration channel 146a from line 146 and aspiration pump 126. AVS module 150 can be autonomous, with a processor inside AVS 150 that receives and processes sensor readings and commands activation of module 150, or module 150 can be commanded from processor 138. To avoid vacuum surge inside the eye, the AVS module 150 very quickly (e.g., within a few tens of milliseconds) cuts off the aspiration line 146. However, blocking aspiration indefinitely by closing the valve of the AVS module 150 compromises “followability.” Followability is defined as the ability to attract cataract pieces to the phacoemulsification tip. Sufficient followability is important for efficient and safe phacoemulsification. An overly reactive system (or user) regarding vacuum surges as the tip is placed or moved within the capsular bag causes the system to fully interrupt suction (e.g., using the AVS mechanism) in cases of IOP drop, and such overreaction to AVS operation degrades followability.

To overcome or minimize the effects of a vacuum surge while maintaining acceptable followability, system 110 allows physician 115 to select a followability level. The processor activates AVS module 150 with a waveform predefined according to the selected followability.

In the example of FIG. 1, user interface 140 and display 136 may be integrated into a touch screen graphical user interface (GUI). Display 136 shows a followability level scale 137 that physician 115 can use to select a given discrete level. For each user there exists some preferable followability level. In the example of FIG. 1, display 136 shows a discrete followability level scale 137. The physician may therefore adjust the followability level, as needed, based on experience. For example, a physician who wishes less disrupted suction power occurring by any possible false positive AVS activation events may tolerate less AVS protection, by selecting a higher followability level on the GUI.

In other examples, the user may adjust the followability level with a virtual slide ruler, enter a numerical value, or make a verbal instruction.

It should be appreciated that the term anti-vacuum surge (AVS) module is used herein to refer to systems for preventing a sudden outflow of fluid from the eye chamber after an occlusion break due to vacuum build up in the aspiration line caused by an occlusion at the distal end of the needle, but such systems may be referred to equivalently by other terminology such as chamber stability/stabilization system (CSS).

As noted above, processor 138 may control the flow rate of irrigation pump 124 and/or aspiration pump 126, where one of the software modules running in processor 138 is a proportional-integral-derivative (PID) controller 114. Processor 138 estimates the IOP using readings from the irrigation pressure sensor 127 and an optional empirical offset (if the irrigation pressure is measured at the proximal end of handpiece 112). Readings of sensor 123 give the vacuum level (also called sub-pressure) inside the aspiration channel.

Sensors 127 and 123 may be any sensor known in the art, including, but not limited to, a vacuum sensor or flow sensor. The sensor measurements (e.g., of pressure, vacuum, and/or flow) may optionally be taken close to the distal end of the handpiece where the irrigation outlet and the aspiration inlet are located, so as to provide processor 138 with an accurate indication of the actual measurements occurring within an eye, thus providing a short response time to a control loop comprised in processor 138.

In an example, the same pressure sensor model is used to measure irrigation pressure and aspiration sub-pressure, using different sensor settings/calibrations.

As further shown, phacoemulsification probe 112 includes a piezoelectric crystal 155, coupled to a horn (not shown), that drives needle 116 to vibrate in a resonant vibration mode that is used to break a cataract into small pieces during a phacoemulsification procedure. Console 128 comprises a piezoelectric drive module 130, coupled with the piezoelectric crystal, using electrical wiring running in cable 133.

Processor 138 may receive user-based commands via a user interface 140, which may include setting a vibration mode and/or frequency of the piezoelectric crystal, and setting or adjusting an irrigation and/or aspiration rate of the irrigation pump 124 and aspiration pump 126. Optionally, user interface 140 includes a foot pedal. Processor 138 may receive user-based commands via a user interface 140, which may include stroke amplitude settings for the needle 116 and commands for initiation of irrigation and/or aspiration. In an example, the physician uses a foot pedal (not shown) as a means of control. For example, a foot pedal may have a treadle that is moveable in a pitch direction and the available pitch travel may be divided into multiple functionality zones or positions. Foot pedal position one activates only irrigation, foot pedal position two activates both irrigation and aspiration, and foot pedal position three adds needle 116 vibration. Additionally, or alternatively, processor 138 may receive user-based commands from controls located in a handle 121 of probe 112.

Some or all of the functions of processor 138 may be combined in a single physical component or, alternatively, implemented using multiple physical components. These physical components may comprise hard-wired or programmable devices, or a combination of the two. In some examples, at least some of the functions of processor 138 may be carried out by suitable software stored in a memory 135 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 is simplified for clarity of presentation. For example, the disclosed AVS scheme may be applied using alternative or additional control devices of the system, such as other valves (e.g., a bypass valve).

This particular configuration of system 110 is shown by way of example, in order to illustrate certain problems that are addressed by examples of the present invention and to demonstrate the application of these examples in enhancing the performance of such a phacoemulsification system. Examples of the present invention, however, are by no means limited to this specific sort of example phacoemulsification system, and the principles described herein may similarly be applied to other sorts of phacoemulsification or other suitable types of surgical systems.

As provided substantially above, the surgical environment may include a surgical media system that interfaces with components of the system 110. The surgical media system may be implemented as standalone equipment (e.g., physically separate from the system 110), as hardware integral within one or more components of the system 110, or via software executed by processing means of the system 110. One problem with the surgical media system is that a standalone implementation may require expensive hardware and may only be available in a wired configuration. For example, operating room personnel may be required to connect surgical media system equipment to a video converter connected to a video source (e.g., a surgical microscope and/or camera) and to a surgical system with video cable and serial cable to set-up the surgical media system for use with a single external monitor. A wired configuration may be disadvantageous in a surgical environment, as wires may obstruct movement of operating room personnel and equipment within the operating room and may harbor contaminants. Another related problem is that the overlay may only be available to a limited number of external devices, whereas it may be advantageous to stream the video feed with or without the overlay across multiple platforms.

Yet another problem arises when the performance of the system suffers due to graphical processing constraints. For example, the resolution of a combined video feed and overlay may be limited. In addition, the system may perform poorly if the surgical media system executes additional streaming programs while processing the video feed and overlay.

A next-generation surgical media system as proposed herein may address at least the above-described problems. As is described in further detail in paragraphs that follow, a surgical media system according to at least one of the proposed examples supports features to improve usability of the surgical media system and improve the operating room personnel workflow during surgery. With an improved system architecture, software enhancements, and wireless interface capabilities, a new, lower-cost surgical media system can meet existing surgical media system requirements while supporting new and useful features that better serve the needs of operating room staff. A surgical media system according to the examples described herein may provide such features through an all-in-one graphics, hardware, and software solution, or via standalone software packages.

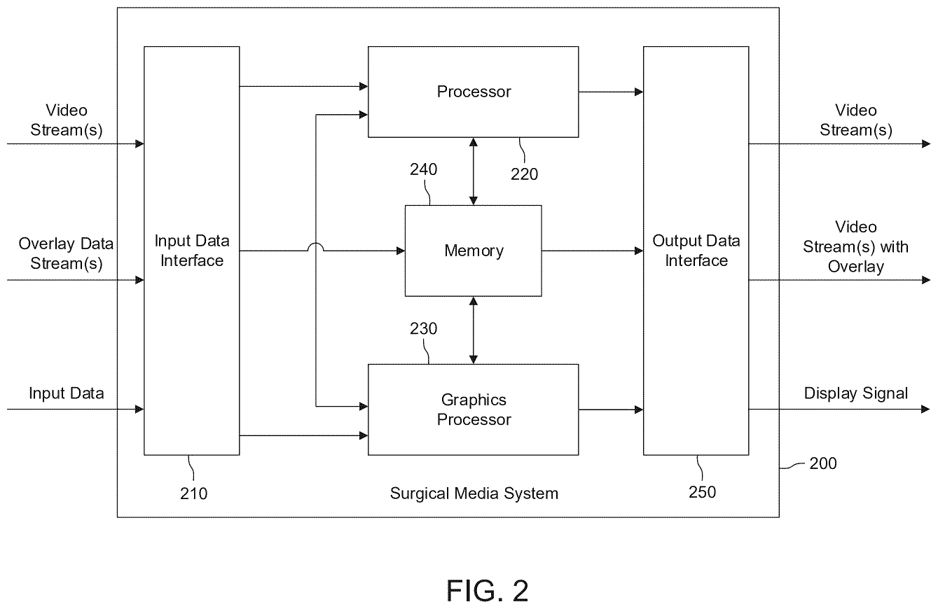

FIG. 2 is a functional block diagram illustrating components and interfaces of a surgical media system in a surgical environment. As illustrated in FIG. 2, the surgical media system 200 includes an input data interface 210, a processor 220, a graphics processor 230, one or more memory devices 240, and an output data interface 250.

The input data interface 210 and the output data interface 250 include hardware for receiving and sending video data, overlay information, and input data. The data interfaces 210 and 250 are configured to allow packets and signals to be transferred among the surgical media system 200 and external devices. In particular, the input and output data interfaces 210 and 250 (also referred to collectively herein as data interfaces) enable communication with other devices in the surgical environment via wired and/or wireless means. The input and output data interfaces may be configured to receive and send electronic, electromagnetic, optical, radio frequency (RF), or other signals as will be apparent to persons having skill in the relevant art. The signals travel via a communications path that may be specifically configured to carry the signals and may be implemented using wire, cable, fiber optics, a phone line, a cellular phone link, a radio frequency link, etc. Though not shown, the input and output data interfaces 210 and 250 may include one or more antennas coupled to a radio transceiver or an RF front end (including, for example, mixers, amplifiers, oscillators, filters (e.g., digital signal processors), and/or analog-to-digital converters (ADCs)). In some examples, the input data interface 210 may include one or more antennas coupled with a radio receiver, and the output data interface 250 may include one or more antennas coupled with a radio transmitter. It should be noted that although the input data interface 210 is illustrated separately from the 250, in some examples, the interfaces need not be implemented separately in hardware. For example, an input data interface 210 that provides for reception of signals via a wireless transceiver may share such hardware resources with the output data interface 250. Those of ordinary skill in the art will appreciate that a wireless interface may be implemented in accordance with a variety of different protocols including cellular protocols such as those set forth in Third Generation Partnership Project (3GPP) Long-Term Evolution (LTE) and New Radio (NR) specifications, Institute of Electrical and Electronics Engineers (IEEE) 802.11 Wireless Local Area Network (WLAN) specifications, Bluetooth®, among other protocols.

In some examples, the input and output data interfaces 210 and 250 may be implemented virtually via software. For example, instead of requiring that a discrete hardware receiving a video data stream via a cable plugged into a serial port, surgical media system software may be written such that a logical entity, providing surgical media system functionality as described herein (also referred to herein as “surgical media system software”), communicates with other logical or physical entities or endpoints in a network via the input and/or output data interfaces 210 and 250. The logical surgical media system may communicate packetized data with other entities or endpoints in a public network using, for example, internet protocol (IP) infrastructure or with other entities or endpoints in an intranet (i.e., a private network). An intranet may be a computer network administered by an information technology (IT) department an organization such as a healthcare provider or surgical center and providing communication and computing services between approved entities or endpoints. The intranet may serve devices operating in an operating room environment as well as external devices connected to the devices within the operating room environment. In other examples not depicted in FIG. 2, the functionality of the input and output data interfaces 210 and 250 may be realized by a single interface that receives, processes, and sends input and output data.

The processor 220 may be a general purpose programmable processor or controller for executing application programming or instructions. In accordance with at least some examples, the processor 220 may include multiple processor cores, and/or implement multiple virtual processors. In accordance with still other examples, the processor 220 may include multiple physical processors. In one example, the processor 220 includes a specially configured application specific integrated circuit (ASIC) or other integrated circuit, a digital signal processor, a controller, a hardwired electronic or logic circuit, a programmable logic device or gate array, a special purpose computer, or the like. The processor 220 generally functions to run programming code or instructions implementing various functions of the surgical media system 200. The processor 220 is configured to interface with one or more memory modules, such as memory 240, which may include internal and/or external storage spaces for data, executable instructions, and other content communicated via or necessary for communication. Though not depicted in FIG. 2, the surgical media system may in some examples include multiple processors.

Memory 240 may include one or more memory devices (e.g., random access memory, read-only memory, etc.). The memory 240 and the one or more additional memories may be read from and/or written to in a well-known manner. In an example, the memory 240 and the one or more additional memories may be non-transitory computer readable recording media. surgical media system functionality may be realized by the processor 220 operating executable code stored in the memory 240. The processor 220 and the memory 240 may be configured to store video data received via an input data stream. The processor 220 and the memory 240 may be configured to store overlay information received via an input data stream. In addition, the processor 220, the graphics processor 230, and the memory 240 may be configured to store an output video stream including overlay information. The present design may be configured to store the received video data stream as analog video, digital video, digital data, or any combination thereof.

Data stored in the memory 240 may be stored on any type of suitable computer readable media. Computer readable media may include any non-volatile or non-transitory media, such as optical storage (e.g., a compact disc, digital versatile disc, Blu-ray disc, etc.), magnetic tape storage (e.g., a hard disk drive (HDD)), or a solid-state drive (SSD). An operating system can be stored in the memory 240.

Memory semiconductors (e.g., DRAMs, etc.) may be a means for providing software to the surgical media system 200. Computer programs (e.g., computer control logic) may be stored in the memory 240. Computer programs may also be received via the input data interface 210. Such computer programs, when executed, may enable the surgical media system 200 to implement methods as discussed herein. In particular, the computer programs stored on a non-transitory computer-readable medium, when executed, may enable a processor to implement functionally as discussed herein. When any aspect of the present disclosure is implemented using software, the software may be stored in a computer program product or non-transitory computer readable medium and loaded into the surgical media system using a removable storage drive or the input data interface 210.

The graphics processor 230 provides encoding/decoding and/or compression/decompression functionality for receiving and managing video stream information. Similar to the processor 210, the graphics processor 230 may include multiple processor cores, and/or implement multiple virtual processors. The graphics processor 230 may include multiple physical processors. In one example, the graphics processor 230 includes a specially configured application specific integrated circuit (ASIC) or other integrated circuit, a digital signal processor, a controller, a hardwired electronic or logic circuit, a programmable logic device or gate array, or a special purpose computer. It should be noted that although the graphics processor 230 is illustrated separately from the processor 220, in some examples, the graphics processor 230 need not be implemented in discrete graphics hardware, and any such graphics processing functionality may be provided by the processor 220 or remotely by an endpoint logically coupled with other elements of the surgical media system 200 as described herein. For example, graphics processing functionality may be provided externally such as via a cloud-based computing application (e.g., an application hosted by a networked server).

The graphics processor 230 as shown in FIG. 2 enables decompression and/or decoding of analog and/or digital information generated by another device within the surgical environment, such as a video source (e.g., a surgical microscope, which may be coupled with a camera), or other types of sensors. Examples of video data formats as may be supported by the surgical media system 200 may include national television system committee (NTSC), phase alternating line (PAL), high definition television (HDTV), séquentiel couleur à mémoire (SECAM), among others.

The graphics processor 230 may be operable to combine graphical images from the video stream received by the input data interface 210 or from the memory 230 with data from the received overlay stream to form a combined video stream and overlay output stream. An encoded video stream may be generated by the graphics processor 230 and sent, via the output data interface 250 to a display device within the surgical environment or integral with the surgical media system 200. Any encoding/decoding and compression/decompression may be performed on the basis of various formats (e.g., audio, video, and data). The graphics processor may provide for encryption mechanisms that impart confidentiality of all the data received or transmitted by other devices within the surgical environment.

In generating the encoded video stream, the processor 220 and the graphics processor 230 manage the data from the video stream as well as data from the overlay information stream in real-time. Managing video and overlay information may include operations such as recording, playback, editing, previewing and bookmarking. Examples of overlay information supported by the present design may include, but are not limited to, vacuum, aspiration, and/or irrigation flow, ultrasonic power, foot pedal position (e.g., foot pedal position 1 (FP1), foot pedal position 2 (FP2), and foot pedal position 3 (FP3)), installed accessories, operation mode, or other data generated from a surgical console during the surgical procedure.

Managing the multimedia data, i.e., the video data stream, may require the surgical media system to synchronize the surgical parameters and the feedback and/or sensor data received in the overlay information that are used or observed contemporaneously at the time the video data is captured. When synchronized, the surgical media system produces an output video data stream in combination with the corresponding parameters and instrument settings at each instant during the procedure, for example, as a graphical video overlay.

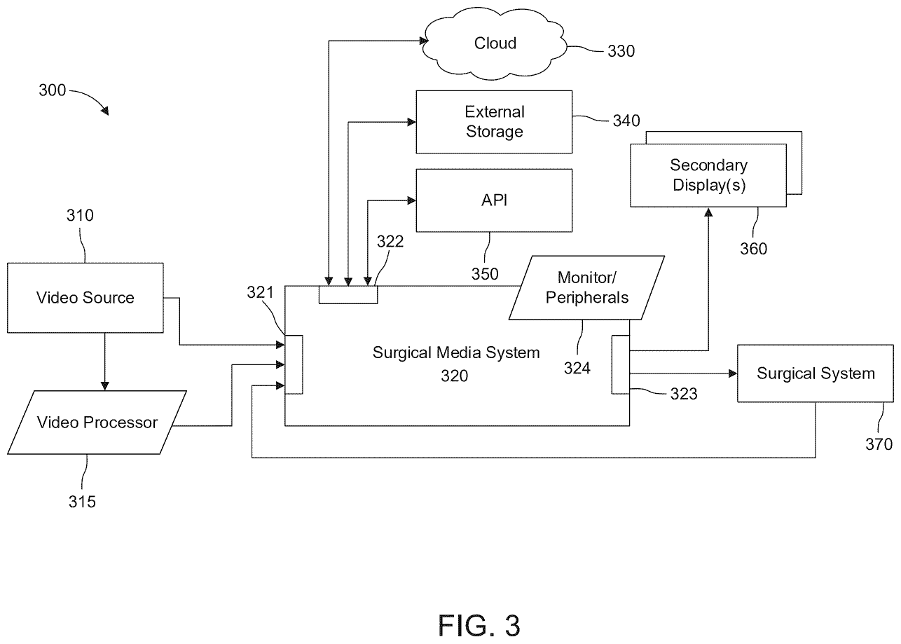

FIG. 3 is a diagram illustrating devices of an exemplary system in which the surgical media system operates. As shown in FIG. 3, the surgical environment or system 300 includes a video output device 310, the surgical media system 320, one or more secondary displays 360, and a surgical system 370. The surgical system 370 may include devices and instruments used by the surgeon to carry out the procedure. For example, in the context of a phacoemulsification procedure, the surgical system 370 refers to a phacoemulsification console and associated handpiece, accessories, (e.g., tubing, ultrasonic tip, sleeve, etc.), foot pedal, and consumables. Although examples are provided herein with reference to phacoemulsification systems and procedures, it should be understood to those of ordinary skill in the art that the surgical media system and surgical environment as described herein may be implemented within other ocular surgical systems or systems used to carry out other types of procedures (e.g., for vitrectomy, diathermy, surgical vision correction, etc.) that include hardware or functionality for capturing video streams and surgical parameters.

In the example shown in FIG. 3, the video source 310 is a surgical microscope, though the video source 310 may, in other examples not shown, include one or more types of devices used to record, capture, sense, scan, process, encode, decode, and/or transcode video and/or audio data. By way of example, the video source may include one or more surgical microscopes, cameras, sensors, sensors, video processors, computers (e.g., general purpose computers and/or special computers), and/or hardware or software having media capture, media stream management, and/or video editing capabilities. The video source 310 may include a single device, or alternatively, multiple devices (i.e., multiple physical and/or logical devices) linked to one another. In some examples, the video source is composed of a surgical microscope, a camera, and one or more intermediary devices (e.g., video capture cards, stream management hardware), which together process captured video for delivery to one or more displays. In some examples, the video source 310 is composed of a device and one or more software modules that may be configured to process, encode, decode, and/or transcode the captured media. In the case the video source 310 includes multiple devices, wired or wireless, networked, or virtualized interfaces may facilitate the exchange of data between any of the devices that carry out video capture, processing, encoding, decoding, and/or transcoding.

In the example shown in FIG. 3, the video source 310 is a surgical microscope used by a surgeon to view a patient's eye during a surgical procedure. In some examples, a camera video recorder may be integrated within the surgical microscope or mounted on and connected with the surgical microscope. The camera video recorder may be a microscopic camera. This may allow the surgeon to record the surgical procedure, if so desired. Optionally, the video source 310 (i.e., the surgical scope and camera video recorder) is coupled with an external video processor 315, which may in turn processor and send a video stream captured by camera video recorder to the surgical media system 320.

Video output from the video source 310 may be transmitted to the surgical system 370 and to one or more secondary displays 360. The surgical system 370 may provide a display (e.g., a primary display) that is easily viewed and accessible by operating room personnel who are involved in the procedure. The secondary displays 360 traditionally used in surgical environments are flat screen television panels mounted in the operating room, though the term “secondary display” used herein may refer to any display device such as a projector, smartphone, tablet, personal computer, wearable, or augmented reality (AR) device. The secondary displays 360 may be provided for the benefit of those in or nearby the operating room, such as other surgeons, residents, scrub technicians, nurses, and other staff who also may aid the surgeon so they may see the live video feed of the surgery. As shown in FIG. 3, the output data interface 323 provides for unidirectional communication with the secondary displays 360. However, those of ordinary skill in the art will easily understand that in other examples not depicted, the surgical media system 320 may be configured with an interface that allows for bi-directional communication with the secondary displays 360.

As shown in FIG. 3, optionally, the surgical media system 320 may be coupled with a monitor 324. For hardware-based implementations of the surgical media system as shown in FIG. 3, the monitor 324 may enable users to directly configure the surgical media system, such as via a graphical user interface and input devices (not illustrated herein). In some examples, the surgical media system 320 may be implemented logically as software executed by a computer (not illustrated) that is coupled with input devices and a display device operable to display a graphical user interface. The computer is configured to receive user inputs to configure parameters for processing a video stream (e.g., from the surgical microscope 310, external video processor 315, or from local or external storage) and/or for generating a combined video/overlay stream.

As described in paragraphs above, the surgical media system 320 is configured with video processing capabilities that enable the surgical media system 320 to manipulate surgical data to be overlaid upon a live video stream. The surgical media system 320 is configured with an input data interface 321 and an output data interface 323. Additionally, the surgical media system is configured with a communication interface 322 that enables bi-directional communication with external applications and storage devices. Similarly, as described in paragraphs above, the input data interface 321, the output data interface 323, and the communication interface 322 may be implemented virtually via software. Alternatively, or additionally, although the input data interface 321, the output data interface 323, and the communication interface 322 are illustrated separately in FIG. 3, any of the input data interface 321, the output data interface 323 and/or the communication interface 322 may be implemented via the same hardware, with data being routed between different interconnected components through circuitry in a serial or parallel fashion.

It should be understood that any of the components illustrated in FIG. 3 with adjoining arrows may be coupled directly through wired or wireless means. It should be further understood that any of the components illustrated in FIG. 3 (whether illustrated with adjoining arrows or not) may be coupled indirectly via one or more of the input data interface 321, the output data interface 323 and/or the communication interface 322. In some examples, any of the components illustrated in FIG. 3 may be coupled directly or indirectly through other components or interfaces not explicitly shown. The input data interface 321, the output data interface 323 and/or the communication interface 322 may include, but are not limited to the following: serial and/or parallel interfaces such as Universal Serial Bus (USB), Institute of Electrical and Electronics Engineers (IEEE) 1394, Serial AT Attachment (SATA), Peripheral Component Interconnect (PCI), or Parallel ATA (PATA); printed circuit board (PCB) traces; wired or wireless network interfaces such as Ethernet, Wi-Fi (IEEE 802.11), Bluetooth®; Cellular (e.g., 3GPP LTE/NR); sensor interfaces providing analog data, such as voltage or current signals; display interfaces such as High-Definition Multimedia Interface (HDMI) or DisplayPort; or memory interfaces such as Double Data Rate (DDR) or Flash.

In an example, users of applications such as Johnson & Johnson's Cataract Analysis and Settings Application (CASA) may communicate with the surgical media system 320 (e.g., via an application programming interface (API) 350 provided by the communication interface 322). This may enable users to request and retrieve video streams and surgical data from the surgical media system 320. In response, the surgical media system may forward the requested video stream(s) and surgical data to the users (e.g., to the users of CASA, via the API 350).

Via the communication interface 322, the surgical media system 320 may also have access to cloud computing resources for computer vision and artificial intelligence. For example, to support graphical enhancements as will be described in paragraphs below, the surgical media system 320, may request application support from a cloud computing platform 330 (e.g., for image enhancement). This may allow the surgical media system 320 to offload processing burdens and/or reduce the level of processing capability required for the surgical media system hardware, which may be instrumental in reducing the complexity and cost of the surgical media system. The surgical media system 320 may send optimized video and/or overlay information to be included in a video stream to the surgical system 370 and/or the secondary display(s) 360. The cloud computing platform may be, for example, a networked server that hosts an application providing functionality of the surgical media system.

In addition to, or as an alternative to storing data in memory arrays housed within the surgical media system 320 itself, the surgical media system 320 may be configured to store video stream and overlay information in external storage 340 via the communication interface 322. The external storage 340 may advantageously provide backup protection and/or security measures. The surgical media system 320 may access the external storage 340 via the communication interface 322 both to retrieve stored video and overlay information and to upload new data.

Although the surgical media system 320 as shown in FIG. 3 is illustrated separately from the surgical system 370, those of skill in the art will appreciate that a surgical console, or another device within the surgical environment 300, may be configured to execute software providing substantially the same surgical media system functionality as is described herein. For instance, the surgical system 370 may include some or all of the components illustrated in the functional block diagram of FIG. 2, which may be configured to receive video data and overlay/input data streams, process said video data and overlay/input data streams to generate, store, and/or display signals and video streams with or without overlay information. Video data and overlay/input data may be processed, sent, and/or received between components of the surgical system 370, consistent with the illustration of FIG. 3 The surgical system 370 may be coupled directly with the video source 310 and/or via a video processor similarly to the surgical media system 320 illustrated in FIG. 3. A surgical system 370 that implements such surgical media system functionality may also interface with secondary displays 360, cloud-based systems 330, external storage 340, and other applications via wired or wireless interfaces.

The surgical media system and system architectures illustrated in FIGS. 2 and 3 may enable support for various new features that are useful in a surgical environment, such as adaptive streaming within both the internet and intranet, wireless casting of video streams, post-procedure review and analysis, Although the methods provided herein may be described with reference to surgical media system hardware, persons of ordinary skill in the art will appreciate that any of the methods described herein may be implemented across a wide variety of platforms, and the surgical media system may be implemented logically through a standalone software package (e.g., running on a device such as a surgical console, general-purpose computer, mobile device, or network server).

The surgical media system or surgical media system software may be configured to adaptively change resolution and image quality parameters for a given video stream. The surgical media system may, for example, determine that operating room conditions (such as ambient lighting conditions, color temperature, etc.), necessitate adjustment to image quality parameters such as brightness, contrast, color adjustments, etc. The surgical media system or surgical media system software may obtain information about the operating room conditions from a variety of sources. For example, although not illustrated in the functional block diagram and system architecture of FIGS. 2 and 3, the surgical media system or surgical media system software may be in communication with one or more sensors that enable the surgical media system to obtain information about the operating room conditions.

Alternatively, or additionally, the surgical media system or surgical media system software may receive data from other devices in the surgical environment (e.g., from the surgical system, from the video source, or from receive devices to which video data may be streamed) indicating operating room conditions. Alternatively, or additionally, the surgical media system or surgical media system software may be configured to receive user input data indicating the operating room conditions or preferences (e.g., accessibility options) that inform how the surgical media system or surgical media system software adapts resolution and image quality parameters.

In some examples, the surgical media system or surgical media system software may adjust resolution and/or image quality parameters for a given video stream based on an amount of bandwidth. The surgical media system or surgical media system software may determine or monitor the bandwidth or a maximum possible data rate for a given path, for example, a link or a path between the surgical media system or a device implementing surgical media system software and another receive device. For example, if the bandwidth drops drastically when switching a stream from a wired interface to a wireless interface, or if the surgical media system or surgical media system software begins processing and sending additional video streams to other receive devices, the bandwidth for each stream may drop. The surgical media system or surgical media system software may in turn reduce resolution or image quality parameters to compensate for the decreased bandwidth. In another example, the surgical media system or surgical media system software may be configured to adaptively change resolution and/or image quality parameters based on the processing load experienced at the surgical media system and/or the receive device. In another example, the surgical media system or surgical media system software may adjust graphics processing parameters to increase a compression ratio for one or more of the video streams. For example, the surgical media system or surgical media system software may increase or decrease a compression ratio for one or more of the video streams based on the available bandwidth or based on the quality of a link or a path between the surgical media system or a device implementing surgical media system software and a receive device.

Additionally, or alternatively, when sending a video stream to a receive device using a wireless interface, the surgical media system or surgical media system software may be configured to perform resolution and/or image adjustment based on observed or expected channel conditions. In another example, the surgical media system or surgical media system software may adjust resolution and image quality parameters based on a latency requirement. For example, the surgical media system, a receive device, surgical system, or cloud-based platform in communication with the surgical media system or surgical media system software may be configured to adjust processing parameters to ensure latency for a given stream remains at an acceptable level. The surgical media system or surgical media system software may be configured to lower resolution and/or image quality parameters to reduce the amount of time required to render a stream. In another example, the surgical media system or surgical media system software may adjust resolution and image quality parameters based on a capability (e.g., a display capability) of the receive device to which video data may be streamed, such as a supported resolution, color depth, and/or dynamic range. In some examples, the surgical media system may adjust resolution and image quality parameters based on display settings configured at the receive device.

When enhanced with wireless interface capabilities, the surgical media system may be able to send video streams, with or without overlay information, to one or more receive devices without suffering from limitations of physical interfaces such as the number of available physical connectors. The surgical media system may retain the ability to stream video and/or combined video/overlay feeds via wired interfaces.

The surgical media system may send the live video stream or combined video and overlay stream to multiple receive devices via multiple feeds simultaneously. For example, in a surgical environment that includes multiple receive devices providing secondary displays, the surgical media system may establish a link with each receive device and transmit separate video or video/overlay streams to each receive device via the output data interface. The surgical media system may separately process each feed according to requirements and parameters specific to each receive device. Video streams sent to multiple different receive devices may include the same or different information. For example, different feeds may provide different video streams, different overlay information, different combined video and overlay streams, or different enhancements of the video streams and/or overlay information described in further detail in paragraphs below.

The surgical media system may be configured to send the video or combined video/overlay to multiple receive devices using a joint stream. For example, the surgical media system may obtain channel resources for sending the joint stream using a broadcast transmission. Alternatively, or additionally, the surgical media system may designate a group of devices that includes at least a portion of the intended receive devices. The surgical media system may then send the stream to the group using a multicast or groupcast transmission.

Methods for processing overlay information and generating a combined video/overlay stream are described herein. Substantially as described above, the surgical media system may provide functionality for processing surgical parameters and other data to derive overlay content. Overlay information may include, but is not limited to: surgical parameters, sensor readings and other surgical data observed or obtained by a surgical system in real-time, data associated with equipment or accessories used to perform the surgical procedure, metadata associated with the surgical procedure (including, e.g., for the surgical environment/operating room, operating room personnel, and patient data), user-entered and/or system generated annotation information, captions, and targeted advertisements. The data associated with equipment or accessories may include, for example, information related to a cassette (e.g., a type of cassette) configured for used by the surgical system, information related to a type or size of tubing used to connect various components of the surgical system, an indication of an active mode (e.g., an active mode of the surgical system), an indication of a handpiece configured for use by the surgical system (e.g., a type of handpiece such as a phacoemulsification handpiece or a vitrector, a type of handpiece corresponding to a particular handpiece manufacturer and/or model, a type of handpiece used for a specific mode within a given surgical procedure (e.g., an irrigation/aspiration (I/A) handpiece as may be used within a phacoemulsification procedure) an indication of a foot pedal configured for used by the surgical system (e.g., a type of foot pedal), an indication of an active foot pedal position, or other information associated with a status of the foot pedal.

In some examples, the foot pedal may be one of multiple types, where the indicated type may correspond to a particular footpedal manufacturer and/or model, capabilities (e.g., wired vs. wireless connectivity with the surgical console, and/or battery-operated vs. corded power). The status of the foot pedal may refer to a battery level, percentage, voltage, or charging status; a signal strength or connection quality such as in the case of a wireless foot pedal, a connection status (i.e., connected or disconnected); whether the foot pedal is in a locked or unlocked state (i.e., locked to prevent physical actuation or movement the foot pedal or other buttons or switches located on the foot pedal, or locked to prevent changes to surgical parameters caused, for example, by inadvertent physical actuation of the foot pedal); or whether a button or switch on the foot pedal has been pressed.

In some examples, the active foot pedal position may refer to a status of the foot pedal or position of a physical treadle on the foot pedal. The foot pedal may have one or more treadles that may be moved or adjusted respective to one or more axes. For example, a treadle may be capable of pitch adjustment around a horizontal axis. When pressure is applied to the pedal, the treadle may pitch or pivot up or down around the horizontal axis, which sends a signal to the surgical console to which the foot pedal is connected. A sensor may sense the position of the treadle as it rotates above the horizontal axis such that an initial range of rotation may correspond to FP1, a subsequent range of rotation corresponds to FP2, and a final range of rotation corresponds to FP3. The surgical console may be configured to, for example, automatically adjust surgical parameters based on the sensed position of the treadle.

In some examples, a physical treadle on the foot pedal may be capable of side-to-side adjustment or yaw around a vertical axis. The treadle may be rotated side-to-side around the vertical axis from a starting position. Upon rotation of the treadle around the vertical axis from the starting position, the foot pedal sends a signal to the surgical console to adjust a surgical parameter or mode. For example, in the case of a phacoemulsification surgical system, the yaw adjustment may activate or deactivate an irrigation/aspiration (I/A) mode in which little or no ultrasonic power is delivered to the phacoemulsification handpiece. In some examples, the foot pedal may provide a mechanism for bias adjustment of the yaw. For instance, a bias adjustment may enable a greater degree of rotation of the treadle from the starting position to either side, or it may cause the adjustment of one or more surgical parameters when the treadle is rotated toward one side to differ from the adjustment of one or more surgical parameters when the treadle is rotated toward the opposite site.

In a specific example pertaining to phacoemulsification procedures, data that may be included in the overlay include surgical parameters such as: fluidics parameters including set points for vacuum/aspiration/power/irrigation/flow, surgical modes, balanced salt solution usage, balanced salt solution bottle height, continuous irrigation, etc. The overlay information may include real-time observed or measured values for vacuum/power/flow, balanced salt solution usage, balanced salt solution bottle height, and/or effective phaco time (EPT). The data may further include an indication of an active mode or step of the procedure such as, but not limited to: sculpt, chop, quadrant, epi (i.e. epinucleus), cortex (i.e. removal of residual cortex), polish (i.e. anterior capsule polishing), or visco (i.e. viscoelastic-removal).

In some cases, the surgical system parameters may include parameters relating to surgical functions other than phacoemulsification. For example, the vitrectomy is one type of ophthalmic procedure performed in the treatment of retinal detachments, retinopathies, complications from cataract surgery, or removal of vitreous hemorrhage, scar tissue, or foreign bodies. In contrast to phacoemulsification in which an ultrasonic handpiece is used, a surgeon performing the vitrectomy uses a pneumatic or electric-driven cutting handpiece (also referred to as a vitrector), with suction to remove vitreous humor from the posterior chamber of the eye. The size (or gauge) of the vitrector may vary, with common sizes including 20-gauge, 23-gauge, 25-gauge, and 27 gauge. The cutting rate of the vitrector may be adjusted by the surgeon and controlled (possibly in conjunction with aspiration levels) using a foot pedal. A system according to one or more of the examples proposed herein may utilize vitrectomy parameters or data associated with any of the above aspects of the vitrectomy procedure to control the console or handpiece during the surgical procedure, or to produce video content including overlay information.

In another example, diathermy is a procedure that involves the use of electrically induced heat to coagulate or cut tissue. In bipolar diathermy, a handpiece includes two closely spaced electrodes that pass current through the tissue. This provides more controlled and localized delivery of heat, making it ideal for delicate areas like the eye. In monopolar diathermy, a single active electrode is used at the surgical site, with a remote return electrode (grounding pad) placed on the patient's body. The current travels from the active electrode, through the body, to the grounding pad. This technique is less commonly used in ophthalmic procedures due to its broader area of effect compared to bipolar diathermy. Diathermy is often employed in retinal detachment surgeries to create chorioretinal adhesions, helping anchor the retina in place. During the procedure, the surgeon may adjust power settings to control the amount of energy delivered to the tissue. The surgeon may also determine a mode (i.e., continuous or pulsed) in which energy is delivered. Some diathermy systems may utilize feedback mechanisms that adjust power settings in real-time based on, for example, tissue impedance. This may help to maintain consistent coagulation while minimizing tissue damage. A system according to one or more of the examples proposed herein may utilize diathermy parameters or data associated with any of the above aspects of the diathermy procedure to control the console or handpiece during the surgical procedure, or to produce video content including overlay information.

An overlay that is superimposed upon a video stream may include all or a subset of available overlay information. The specific data to be included in the overlay may be automatically configured or user-configurable. For example, a surgical media system may be configured with a default set of data to be included in the overlay. Alternatively, or additionally, the surgical media system, or another device connected to the surgical media system, may be configured to accept a user selection designating which overlay information should be included in a combined video/overlay stream. For example, the surgical media system may accept user inputs selecting specific data that should be displayed in the overlay, such as a timestamp and real-time vacuum/aspiration/power/irrigation flow values. The surgical media system may be configured to store profiles, which, when selected, prompt the surgical media system to produce a combined video/overlay stream providing a designated set of the available overlay information.

The overlay information may be graphically presented in a combined video/overlay stream. The graphical presentation may include various graphical elements such as panels, windows, pop-ups, labels, gauges, graphs, and/or buttons, each having characteristics such as shape, size, color, transparency, and spatial positioning within the framing of the combined video/overlay stream. Similar characteristics may apply to text displayed in the overlay. The characteristics of the graphical presentation may be automatically configured by the surgical media system and/or may be user-configurable. The surgical media system may be configured to store user-selectable themes specifying characteristics of the graphical presentation. When selected, the themes may prompt the surgical media system to produce a combined video/overlay stream having the specified characteristics. For example, in some examples, the surgical media system may be configured with a “dark mode” theme that adjusts parameters such as color, brightness, transparency, as well as text characteristics, to accommodate viewing in dim environments or viewing by the visually impaired.

In some examples, the surgical media system may be configured to provide a graphical user interface that enables a user to select and arrange contents for display. The graphical user interface may enable the user to resize, reposition, add, and remove graphical elements. In some examples, the surgical media system may provide for separate selection and arrangement of graphical elements for each individual video/overlay stream that it transmits. The graphical user interface may be accessible to the user via a display and peripheral input devices (e.g., a touchscreen, mouse and keyboard, etc.) coupled with the surgical media system, via a display and peripheral input devices coupled with a receive device that is in communication with the surgical media system, or via a software application made available through an API.

As described in the following paragraphs, surgical media system functionality may be enhanced using artificial intelligence (AI) and/or machine learning (ML) functionality to provide image recognition, event recognition, and image processing capabilities. In hardware-based implementations, the surgical media system itself, or a computer programmed to execute a surgical media system software package, may be programmed to execute AI and/or ML algorithms to aid in the processing of video streams and overlay information. Additionally, or alternatively, AI capabilities may be provided by an external service (e.g., available through a cloud-computing platform) that the surgical media system may interface with or via another endpoint in the same network that provides similar processing resources. In some examples, AI and/or ML algorithms may be used for recognition of anatomical features of a patient or equipment of the surgical system.

In some examples, AI and/or ML algorithms may be used to arrange or rearrange graphical elements of an overlay stream, provide notational information associated with the surgical procedure (e.g., notational information identifying anatomical features of a patient or equipment of the surgical system), and/or ensure that graphical elements or notational information do not overlap with each other or with anatomical features of the patient when a combined video stream and overlay are displayed. In some examples, AI and/or ML algorithms may be used to produce or enhance a video stream, such as by tailoring image quality parameters of the video stream based on characteristics of the environmental of a device that will display the video stream. For instance, if a device is configured to display the video stream while present in the surgical environment (e.g., during the surgical procedure), AI and/or ML algorithms may be used to adjust settings such as brightness and/or contrast based on lighting conditions in the surgical environment.

In some examples AI and/or ML algorithms may be used for processing, encoding, and/or decoding of a video stream. For example, AI and/or ML algorithms may be used for content analysis and prediction to efficiently represent redundant information shared between consecutive frames by encoding only the differences between frames, leading to more efficient compression. AI and/or ML algorithms may be used to enhance the quality of compressed videos. Upscaling techniques may use deep learning models to generate high-resolution frames from low-resolution inputs and improve the visual quality of compressed videos. AI and/or ML algorithms may be used for other purposes such as rate-distortion optimization, noise reduction and artifact removal, adaptive encoding based on characteristics of video content and constraints posed by the hardware involved.

In some examples AI and/or ML algorithms may be used to optimize the delivery of video streams between interconnected devices. In networked systems where a communications interface between two components imposes bandwidth constraints, AI and/or ML may be used to deliver video content over the internet while dynamically adjusting video quality based on the available network bandwidth and device capabilities. For example, a video stream may be encoded at multiple different bitrates, each providing a different video quality. The video stream may be segmented at set intervals. By analyzing an available network bandwidth and device capabilities, an optimal bitrate at which to deliver a video stream to each receive device can be selected for each segment. An AI or ML algorithm that is implemented for such bandwidth-adaptive streaming enhancements may analyze playback quality and network conditions obtained via client-side reporting or server-side analytics. This data can be used to further optimize the adaptive streaming process by driving the selection of the appropriate bitrate.

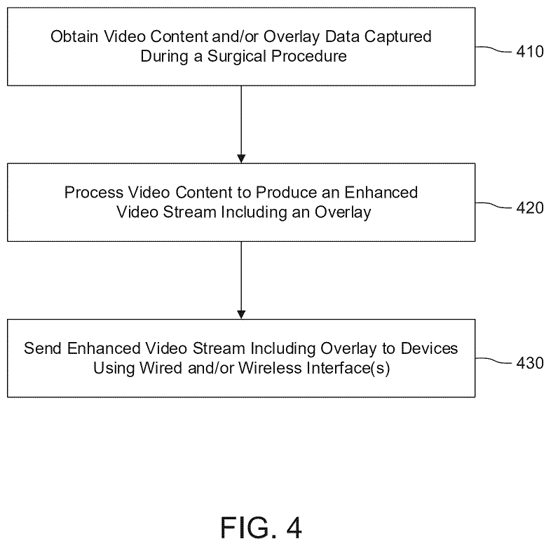

FIG. 4 is a flowchart illustrating steps as may be performed by a surgical media system or by a processor configured to execute surgical media system application(s) for processing video content and overlay information. As shown in FIG. 4, at 410, video and/or overlay information is obtained for processing into a combined video/overlay stream. For example, the video and/or overlay information may be received from a video source and a surgical system respectively. In another example, the video content and/or overlay information may be received from a local or external storage device. At 420, the received video content is processed to generate an enhanced video stream including an overlay.

In some cases, the surgical media system or surgical media system software may process the video content and/or overlay information. In some cases, at least a portion of the received video content and/or the overlay information is provided to a processor (e.g., hardware implemented in or connected to the surgical media system) that provides video stream enhancement functionality. Alternatively, or additionally, at least a portion of the received video and/or overlay information is forwarded to a processing device (e.g., via a data interface such as an API) running an application that supports video stream enhancement. The forwarded video data may include one or more frames of a video stream captured by a video source, such as a surgical microscope. The forwarded video content and/or overlay information may be processed by the processor providing video stream enhancement functionality, or by the processing device that provides video stream enhancement application. The video stream enhancement application may provide image enhancement information to the surgical media system or surgical media system software, which may be used by the surgical media system to produce an enhanced video stream. The received image enhancement information may include video stream data processed by the video stream enhancement application, or information that the surgical media system or surgical media system software processes in conjunction with the video content and overlay information to produce the enhanced video stream. At 430, the surgical media system or surgical media system software may send the enhanced video stream including an overlay to devices using wired and/or wireless interfaces.

In some examples, the surgical media system, surgical media system software, or an application hosted by another network entity may be configured to recognize, track, and/or label objects displayed in the video stream using computer vision and image processing algorithms. Such computer vision and image processing algorithms may include neural network-based and/or non-neural network based algorithms.

In some examples, the surgical media system, surgical media system software, or an application hosted by another network entity may be configured to pre-process the video stream to enhance quality through noise reduction to remove undesired artifacts, normalization of brightness and contrast, color conversion (i.e., from red-green-blue to grayscale) and scaling. Computer vision and/or image processing algorithms may involve feature extraction, in which key features such as edges, corners, or textures are identified and/or extracted, which may reduce the amount of data to be processed. Further processing of the video stream may involve detection and/or segmentation, in which objects are identified or classified, and in which segments of an image of the video stream correspond to specific objects or parts of objects.

In some examples, such as in the context of a phacoemulsification procedure, the surgical media system, surgical media system software, or other application may be configured to identify and track the position of the lens, pupil, iris, cataract, and other anatomical features of the anterior chamber of an eyeball within the video frame. In some examples, the surgical media system, surgical media system software, or other application may be configured to detect movement of the eyeball and process the video and/or overlay information accordingly. For instance, when processing the video stream, the surgical media system, surgical media system software, or other application may be configured to automatically re-center and align the video stream according to the movement of the eyeball, e.g., to keep the eyeball approximately centered within frames of the video stream.

In some examples, the surgical media system, surgical media system software, or other application may be configured to re-arrange overlay information superimposed upon the video stream based on identified anatomical features. For example, having detected and identified anatomical features in the displayed video stream, the surgical media system, surgical media system software, or other application may be configured to adaptively add, re-position, or remove graphical elements (such as gauges, surgical parameters, and other information) within frames of the combined video/overlay stream while tracking movement of the identified anatomical features. This may enhance the viewability of the stream by adding useful overlay information or by presenting graphical elements that highlight objects of importance within the video stream. In addition, this may help to avoid a scenario in which anatomical features of interest are obstructed by graphical elements.

Similarly, as described in paragraphs above, the surgical media system, surgical media system software, or an application hosted by another network entity may be configured to recognize, track, and/or label tools or accessories displayed in the video stream using computer vision and image processing algorithms. The surgical media system, surgical media system software, or other application may be configured to adaptively add, re-position, or remove graphical elements to enhance the presentation of the combined video/overlay stream. In addition to detecting tools or accessories using computer vision and image processing algorithms, objects displayed in the video stream may be identified based on other contextual information, such as information received in overlay information from a surgical system. For example, a phacoemulsification system may have provided data indicating a phacoemulsification tip and sleeve combination being used in the procedure. The surgical media system, surgical media system software, or other application may be configured to identify, or add graphical elements to the combined video/overlay feed labeling the tip and sleeve combination.

In addition, the surgical media system, surgical media system software, or an application hosted by another network entity may be configured to recognize events occurring in the video stream using computer vision, image processing algorithms, and/or contextual information derived from overlay information received by the surgical media system, surgical media system software, or other application. In some examples, the surgical media system, surgical media system software, or other application may be configured to recognize a status or stage of an ongoing procedure. For instance, during a phacoemulsification procedure, the surgical media system, surgical media system software, or other application may be configured to recognize an active handpiece setting (e.g., quadrant, sculpt, or polish) based on movement of the phacoemulsification handpiece and/or based on the tip and sleeve combination being used. In another case, the surgical media system, surgical media system software, or other application may be configured to recognize the occurrence of an occlusion (i.e., a partial or complete restriction of the needle during aspiration of emulsified cataract tissue) or occlusion-break in the phacoemulsification system based on movement of cataract tissue within the anterior chamber and handpiece tip, and based on fluidics parameters and other surgical data provided in real-time by the phacoemulsification system. The surgical media system, surgical media system software, or other application may be configured to include a graphical element or to issue a warning or notification within the combined video/overlay stream indicating a recognized event, status, or stage. The surgical media system, surgical media system software, or other application may be configured to store or send contextual information associated with the recognized events, status, or stage of the procedure. The surgical system may be configured to receive or retrieve such contextual information associated with the recognized event, status, or stage of the procedure and to use the contextual information to control the console and/or handpiece.

In some examples, the surgical media system, surgical media system software, or an application hosted by another network entity may be configured with machine learning capabilities for diagnostic analysis or other optimizations for the surgical procedure. For example, the surgical media system, surgical media system software, or another application may be configured to recognize patterns in video and surgical data and to suggest surgical settings or changes to surgical settings accordingly. The aforementioned machine learning capabilities may be developed through compilation and analysis of training data, which may include video and/or overlay information captured from other surgical procedures (i.e., real-world data including data captured during clinical trials, as well as data captured during studies involving animal models). The surgical media system, surgical media system software, or other application may be configured to store or send suggested surgical settings or other diagnostic information derived using its machine learning capabilities. The surgical system may be configured to receive or retrieve such suggested surgical settings or diagnostic information and to use the suggested surgical settings or diagnostic information to control the console and/or handpiece.

Consistent with any of the above-described examples, a surgical media system or surgical media system software may be configured to store a received video stream, a combined video/overlay stream, or the overlay information locally or at an external storage device. surgical media system software may be configured to provide for post-processing of footage from previously recorded videos and/or images. In addition, the surgical media system or surgical media system software may provide users the ability to access and review the stored video stream, the combined video/overlay stream, or the overlay information. For example, a user may access the stored streams or data via a graphical user interface provided by the surgical media system, a device that executes surgical media system software, or by an application that interfaces with the surgical media system through an API (e.g., CASA). The user may access and receive the stored video and/or overlay stream and data over a wired or wireless interface. The surgical media system or surgical media system software may provide the user the ability to annotate the stream, e.g., by adding captions and notes along with associated embedded time stamps or markers. The user may also be able to add, reconfigure, or remove graphical elements to or from a stored video stream or a combined video/overlay stream. The surgical media system or surgical media system software may be configured to process the stored streams or data according to selections made by the user to produce an enhanced video/overlay stream. For example, the surgical media system or surgical media system software may be configured to optimize the resolution and image quality of the video stream, to identify objects and anatomical features present within the video stream, and/or to optimize the presentation of graphical elements within an enhanced combined video/overlay stream according to one or more of the methods described above. The surgical media system or surgical media system software may be configured to send the new video/overlay stream to one or more receive devices (e.g., a device operated by the user) using a wired or wireless interface as described substantially in paragraphs above.

The present application includes at least the following examples:

### EXAMPLE 1

A method for processing and streaming video content relating to an ophthalmic surgical procedure, the method comprising: receiving video content captured from a video source during the ophthalmic surgical procedure; receiving overlay information from an ophthalmic surgical system coupled with the video source; providing at least a portion of the video content and the overlay information to a processor configured to provide video stream enhancement or to a processing device, via a data interface, that is configured to provide a video stream enhancement application; receiving image enhancement information from the processor or from the processing device via the data interface; processing, based on the received image enhancement information, the video content to produce an enhanced video stream including an overlay representing at least a portion of the overlay information; and sending, using a communication interface, the enhanced video stream to one or more receive devices configured to display the enhanced video stream.

### EXAMPLE 2

The method of example 1, wherein the video content is processed using one or more of an artificial intelligence (AI) or machine learning (ML) algorithm to produce the enhanced video stream.

### EXAMPLE 3

The method according to example 1 or example 2, wherein the overlay information comprises one or more of ophthalmic surgical system parameters used contemporaneously with the capture of the video content or surgical data observed contemporaneously with the capture of the video content.

### EXAMPLE 4

A method according to any one of examples 1-3, wherein the ophthalmic surgical system comprises a console, a handpiece, and a foot pedal configured to control the console and the handpiece, and wherein the ophthalmic surgical system parameters include one or more of: fluidics parameters including one or more of: an intraocular pressure, a vacuum pressure, an aspiration flow rate, an irrigation flow rate or a balanced salt solution status; ultrasonic parameters; vitrectomy parameters; diathermy parameters; an indication of one or more accessories configured for use by the ophthalmic surgical system; an indication of a cassette configured for use by the ophthalmic surgical system; an indication of an active mode of the ophthalmic surgical system; an indication of a handpiece type; an indication of a foot pedal type configured for use by the ophthalmic surgical system; or an indication of a foot pedal status.

### EXAMPLE 5

A method according to any one of examples 1-4 further comprising receiving a request from at least one of the one or more receive devices to obtain the enhanced video stream; transmitting, using the communication interface, the enhanced video stream to the one of the one or more receive devices in response to the received request;

### EXAMPLE 6

A method according to any one of examples 1-5 further comprising adjusting a resolution of the enhanced video stream in accordance with a bandwidth or image quality requirement associated with a link to at least one of the one or more receive devices.

### EXAMPLE 7

A method according to any one of examples 1-6, further comprising: receiving user input data indicating a change in one or more characteristics of a graphical presentation of the overlay; and processing, based on the indicated change in the one or more characteristics of the graphical presentation of the overlay, the video content to produce the enhanced video stream including the overlay.

### EXAMPLE 8

The method of example 7, wherein the change in the one or more characteristics of the graphical presentation of the overlay includes an indication to rearrange graphical elements of the overlay.

### EXAMPLE 9

A method according to any one of examples 7-8, wherein the change in the one or more characteristics of the graphical presentation of the overlay includes an indication to insert one or more graphical elements providing notational information associated with the ophthalmic surgical procedure.

### EXAMPLE 10

A method according to any one of examples 1-9, wherein the image enhancement information includes information associated with one or more recognized anatomical features of a patient or one or more equipment of the ophthalmic surgical system visible within the received video content, and wherein, based on the information associated with one or more recognized anatomical features of a patient or one or more equipment of the ophthalmic surgical system visible within the received video content, the overlay included in the enhanced video stream includes graphical elements identifying the one or more recognized anatomical features of the patient or the one or more equipment of the ophthalmic surgical system,

### EXAMPLE 11

The method of example 10, wherein the enhanced video stream includes one or more graphical elements representing ophthalmic surgical system parameters, and wherein, based on the presence of the graphical elements identifying the one or more recognized anatomical features of the patient or the one or more equipment of the ophthalmic surgical system, the one or more graphical elements representing the ophthalmic surgical system parameters are arranged to not overlap with the graphical elements identifying the one or more recognized anatomical features of the patient or the one or more equipment of the ophthalmic surgical system.

### EXAMPLE 12

A method according to any one of examples 1-11, wherein the enhanced video stream is produced with image quality parameters selected based on ambient lighting conditions of the ophthalmic surgical environment.

### EXAMPLE 13

A method according to any one of examples 1-12, further comprising sending, using the communication interface, another enhanced video stream to another one or more receive devices configured to display the enhanced video stream, wherein the another enhanced video stream includes an overlay that is different from the overlay of the enhanced video stream.

### EXAMPLE 14

A method according to any one of examples 1-13, further comprising receiving at least one of diagnostic information or contextual information associated with a recognized event, status, or stage of an ophthalmic surgical procedure; and controlling one or more of the console or the handpiece based on the received diagnostic information or contextual information associated with the recognized event, status, or stage of the ophthalmic surgical procedure.

### EXAMPLE 15

A surgical media system for processing and streaming video content within an ophthalmic surgical environment, the surgical media system comprising: a processor; at least one data interface; and at least one wireless interface; the at least one data interface configured to receive video content captured from a video source during an ophthalmic surgical procedure; the at least one data interface configured to receive, from an ophthalmic surgical system coupled with the video source, overlay information; the at least one data interface configured to provide at least a portion of the video content and the overlay information to the processor or to a processing device configured to host a video stream enhancement application; the at least one data interface configured to receive image enhancement information from the processor or from the processing device; the processor configured to process, based on the received image enhancement information, the video content to produce an enhanced video stream including an overlay representing at least at portion of the overlay information; and the at least one communication interface configured to transmit the enhanced video stream to a plurality of receive devices configured to display the enhanced video stream.

### EXAMPLE 16

The surgical media system of example 15, wherein the video content is processed using one or more of an artificial intelligence (AI) or machine learning (ML) algorithm to produce the enhanced video stream.

### EXAMPLE 17

The surgical media system of example 15 or example 16, wherein the overlay information comprises ophthalmic surgical system parameters used contemporaneously with the capture of the video content and surgical data observed contemporaneously with the capture of the video content.

### EXAMPLE 18

A surgical media system according to any one of examples 15-17, wherein the ophthalmic surgical system comprises a console, a handpiece, and a foot pedal configured to control the console and the handpiece, and wherein the ophthalmic surgical system parameters include one or more of: fluidics parameters including one or more of: an intraocular pressure, a vacuum pressure, an aspiration flow rate, an irrigation flow rate, or a balanced salt solution status; ultrasonic power parameters; vitrectomy parameters; diathermy parameters; an indication of one or more accessories configured for use by the ophthalmic surgical system; an indication of a cassette configured for use by the ophthalmic surgical system; an indication of a handpiece type; an indication of an active mode of the ophthalmic surgical system; or an indication of a foot pedal status.

### EXAMPLE 19

A surgical media system according to any one of examples 16-18, the at least one data interface configured to receive a request from at least one of the one or more receive devices to obtain the enhanced video stream; the processor and the at least one data interface configured to send the enhanced video stream to the one of the one or more receive devices in response to the received request;

### EXAMPLE 20

A surgical media system according to any one of examples 16-19, the processor configured to adjust a resolution of the enhanced video stream in accordance with a bandwidth or image quality requirement associated with a link to at least one of the one or more receive devices.

### EXAMPLE 21

A surgical media system according to any one of examples 16-20, the at least one data interface configured to receive user input data indicating a change in one or more characteristics of a graphical presentation of the overlay; and the processor configured to process, based on the indicated change in the one or more characteristics of the graphical presentation of the overlay, the video content to produce the enhanced video stream including the overlay.

### EXAMPLE 22

The surgical media system of example 21, wherein the change in the one or more characteristics includes an indication to rearrange graphical elements of the overlay.

### EXAMPLE 23

The surgical media system of example 21 or example 22, wherein the change in the one or more characteristics includes an indication to insert one or more graphical elements providing notational information associated with the ophthalmic surgical procedure.

### EXAMPLE 24

A surgical media system according to any one of examples 16-23, wherein the image enhancement information includes information associated with one or more recognized anatomical features of a patient or one or more equipment of the ophthalmic surgical system visible within the received video content, wherein, based on the information associated with one or more recognized anatomical features of a patient or one or more equipment of the ophthalmic surgical system visible within the received video content, the overlay included in the enhanced video stream includes graphical elements identifying the one or more recognized anatomical features of the patient or the one or more equipment of the ophthalmic surgical system, wherein the enhanced video stream includes one or more graphical elements representing ophthalmic surgical system parameters, and

### EXAMPLE 25

The method of example 24, wherein, based on the presence of the graphical elements identifying the one or more recognized anatomical features of the patient or the one or more equipment of the ophthalmic surgical system, the one or more graphical elements representing the ophthalmic surgical system parameters are arranged to not overlap with the graphical elements identifying the one or more recognized anatomical features of the patient or the one or more equipment of the ophthalmic surgical system.

### EXAMPLE 26

A surgical media system according to any one of examples 16-25, wherein the enhanced video stream is produced with image quality parameters selected based on ambient lighting conditions of the ophthalmic surgical environment.

### EXAMPLE 27

A surgical media system according to any one of examples 16-26, the communication interface configured to send another enhanced video stream to another one or more receive devices configured to display the enhanced video stream, wherein the another enhanced video stream includes an overlay that is different from the overlay of the enhanced video stream.

### EXAMPLE 28

A surgical media system according to any one of examples 16-27, further comprising receiving at least one of diagnostic information or contextual information associated with a recognized event, status, or stage of a surgical procedure; and controlling one or more of the console or the handpiece based on the received diagnostic information or contextual information associated with the recognized event, status, or stage of the surgical procedure.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

What is claimed is:

1. A method for processing and streaming video content relating to an ophthalmic surgical procedure, the method comprising:
receiving video content captured from a video source during the ophthalmic surgical procedure;
receiving overlay information from an ophthalmic surgical system coupled with the video source;
providing at least a portion of the video content and the overlay information to a processor configured to provide video stream enhancement or to a processing device, via a data interface, that is configured to provide a video stream enhancement application;
receiving image enhancement information from the processor or from the processing device via the data interface;
processing, based on the received image enhancement information, the video content to produce an enhanced video stream including an overlay representing at least a portion of the overlay information; and
sending, using a communication interface, the enhanced video stream to one or more receive devices configured to display the enhanced video stream.

2. The method of claim 1, wherein the video content is processed using one or more of an artificial intelligence (AI) or machine learning (ML) algorithm to produce the enhanced video stream.

3. The method of claim 1, wherein the overlay information comprises one or more of ophthalmic surgical system parameters used contemporaneously with the capture of the video content or surgical data observed contemporaneously with the capture of the video content.

4. The method of claim 3, wherein the ophthalmic surgical system comprises a console, a handpiece, and a foot pedal configured to control the console and the handpiece, and wherein the ophthalmic surgical system parameters include one or more of:
fluidics parameters including one or more of: an intraocular pressure, a vacuum pressure, an aspiration flow rate, an irrigation flow rate, or a balanced salt solution status;
ultrasonic parameters;
vitrectomy parameters;
diathermy parameters;
an indication of one or more accessories configured for use by the ophthalmic surgical system;
an indication of a cassette configured for use by the ophthalmic surgical system;
an indication of an active mode of the ophthalmic surgical system;
an indication of a handpiece type;
an indication of a foot pedal type configured for use by the ophthalmic surgical system; or
an indication of a foot pedal status.

5. The method of claim 1, further comprising:
receiving a request from at least one of the one or more receive devices to obtain the enhanced video stream;
transmitting, using the communication interface, the enhanced video stream to the one of the one or more receive devices in response to the received request; and
adjusting a resolution of the enhanced video stream in accordance with a bandwidth or image quality requirement associated with a link to at least one of the one or more receive devices.

6. The method of claim 5, further comprising:
receiving user input data indicating a change in one or more characteristics of a graphical presentation of the overlay; and
processing, based on the indicated change in the one or more characteristics of the graphical presentation of the overlay, the video content to produce the enhanced video stream including the overlay.

7. The method of claim 6, wherein the change in the one or more characteristics of the graphical presentation of the overlay includes an indication to rearrange graphical elements of the overlay.

8. The method of claim 6, wherein the change in the one or more characteristics of the graphical presentation of the overlay includes an indication to insert one or more graphical elements providing notational information associated with the ophthalmic surgical procedure.

9. The method of claim 1, wherein the image enhancement information includes information associated with one or more recognized anatomical features of a patient or one or more equipment of the ophthalmic surgical system visible within the received video content, and wherein, based on the information associated with one or more recognized anatomical features of a patient or one or more equipment of the ophthalmic surgical system visible within the received video content, the overlay included in the enhanced video stream includes graphical elements identifying the one or more recognized anatomical features of the patient or the one or more equipment of the ophthalmic surgical system, wherein the enhanced video stream includes one or more graphical elements representing ophthalmic surgical system parameters, and wherein, based on the presence of the graphical elements identifying the one or more recognized anatomical features of the patient or the one or more equipment of the ophthalmic surgical system, the one or more graphical elements representing the ophthalmic surgical system parameters are arranged to not overlap with the graphical elements identifying the one or more recognized anatomical features of the patient or the one or more equipment of the ophthalmic surgical system.

10. The method of claim 1, wherein the enhanced video stream is produced with image quality parameters selected based on ambient lighting conditions of the ophthalmic surgical environment.

11. The method of claim 1, further comprising sending, using the communication interface, another enhanced video stream to another one or more receive devices that are configured to display the enhanced video stream, wherein the another enhanced video stream includes an overlay that is different from the overlay of the enhanced video stream.

12. A surgical media system for processing and streaming video content relating to an ophthalmic surgical procedure, the surgical media system comprising:
a processor;
at least one data interface; and
at least one communication interface;
the at least one data interface configured to receive video content captured from a video source during an ophthalmic surgical procedure;
the at least one data interface configured to receive, from an ophthalmic surgical system coupled with the video source, overlay information;
the at least one data interface configured to provide at least a portion of the video content and the overlay information to the processor or to a processing device configured to host a video stream enhancement application;
the at least one data interface configured to receive image enhancement information from the processor or from the processing device;
the processor configured to process, based on the received image enhancement information, the video content to produce an enhanced video stream including an overlay representing at least at portion of the overlay information; and
the at least one communication interface configured to transmit the enhanced video stream to a plurality of receive devices configured to display the enhanced video stream.

13. The surgical media system of claim 12, wherein the video content is processed using one or more of an artificial intelligence (AI) or machine learning (ML) algorithm to produce the enhanced video stream.

14. The surgical media system of claim 13, wherein the overlay information comprises ophthalmic surgical system parameters used contemporaneously with the capture of the video content and surgical data observed contemporaneously with the capture of the video content.

15. The surgical media system of claim 14, wherein the ophthalmic surgical system comprises a console, a handpiece, and a foot pedal configured to control the console and the handpiece, and wherein the ophthalmic surgical system parameters include one or more of:
fluidics parameters including one or more of: an intraocular pressure, a vacuum pressure, an aspiration flow rate, an irrigation flow rate, or a balanced salt solution status;
ultrasonic power parameters;
vitrectomy parameters;
diathermy parameters;
an indication of one or more accessories configured for use by the ophthalmic surgical system;
an indication of a cassette configured for use by the ophthalmic surgical system;
an indication of a handpiece type;
an indication of an active mode of the ophthalmic surgical system; or
an indication of a foot pedal status.

16. The surgical media system of claim 13, the at least one data interface configured to receive a request from at least one of the one or more receive devices to obtain the enhanced video stream;
the processor and the at least one data interface configured to send the enhanced video stream to the one of the one or more receive devices in response to the received request; and
the processor configured to adjust a resolution of the enhanced video stream in accordance with a bandwidth or image quality requirement associated with a link to at least one of the one or more receive devices.

17. The surgical media system of claim 16, the at least one data interface configured to receive user input data indicating a change in one or more characteristics of a graphical presentation of the overlay; and
the processor configured to process, based on the indicated change in the one or more characteristics of the graphical presentation of the overlay, the video content to produce the enhanced video stream including the overlay.

18. The surgical media system of claim 17, wherein the change in the one or more characteristics of the graphical presentation of the overlay includes an indication to rearrange graphical elements of the overlay.

19. The surgical media system of claim 17, wherein the change in the one or more characteristics of the graphical presentation of the overlay includes an indication to insert one or more graphical elements providing notational information associated with the ophthalmic surgical procedure.

20. The surgical media system of claim 13, wherein the image enhancement information includes information associated with one or more recognized anatomical features of a patient or one or more equipment of the ophthalmic surgical system visible within the received video content, wherein, based on the information associated with one or more recognized anatomical features of a patient or one or more equipment of the ophthalmic surgical system visible within the received video content, the overlay included in the enhanced video stream includes graphical elements identifying the one or more recognized anatomical features of the patient or the one or more equipment of the ophthalmic surgical system, wherein the enhanced video stream includes one or more graphical elements representing ophthalmic surgical system parameters, and wherein, based on the presence of the graphical elements identifying the one or more recognized anatomical features of the patient or the one or more equipment of the ophthalmic surgical system, the one or more graphical elements representing the ophthalmic surgical system parameters are arranged to not overlap with the graphical elements identifying the one or more recognized anatomical features of the patient or the one or more equipment of the ophthalmic surgical system.

21. The surgical media system of claim 20, wherein the enhanced video stream is produced with image quality parameters selected based on ambient lighting conditions of the ophthalmic surgical environment.

22. The surgical media system of claim of claim 13, the communication interface configured to send another enhanced video stream to another one or more receive devices that are configured to display the enhanced video stream, wherein the another enhanced video stream includes an overlay that is different from the overlay of the enhanced video stream.
